(19) United States
(12) Patent Application Publication
(10) Pub. No.: US 2025/0221692 A1
(43) Pub. Date: Jul. 10, 2025

(54) CERVIX SAMPLER, OPERATING METHOD AND KIT THEREOF

(71) Applicant: HKDR LIMITED, Hong Kong (CN)

(72) Inventor: Chi Yin Natural Chan, Hong Kong (CN)

(21) Appl. No.: 18/672,059

(22) Filed: May 23, 2024

(30) Foreign Application Priority Data
Jan. 4, 2024 (CN) 202410016054.0

(51) Int. Cl. A61B 10/02; A61B 10/00

(52) U.S. Cl. CPC A61B 10/0291; A61B 2010/0074; A61B 2010/0216

(57) ABSTRACT

The present application discloses a cervix sampler, and an operating method and a kit thereof. The sampler includes a housing, the housing including a housing side wall and a closed housing distal wall, the housing side wall including a first notch, and the first notch being formed close to the housing distal wall; a sampling brush configured to be accommodated in the housing, at least a part of the sampling brush being exposed, in at least one state of use, to a position where the first notch is located; and a shaft including a shaft distal end and a shaft proximal end, the shaft distal end being connected to the sampling brush, the shaft proximal end further including a manipulator, and the manipulator, operated by an external force, driving the shaft and the sampling brush to rotate in a circumferential direction, so that the sampling brush acquires biological tissue in a state of use by being in contact with and rubbing against a target sample near the first notch. The sampler can be conveniently placed in a target position so as to obtain sample tissue. The collected biological tissue may be cervix cells, which can be used for papanicolaou (PAP)/human papillomavirus (HPV) Co-testing for cervix cancer screening.

### TECHNICAL FIELD

The present invention relates to the technical field of cervical medical products, and in particular to a cervix sampler, and an operating method and a kit thereof.

### BACKGROUND

In the prior art, cervix sampling products are inconvenient to use, require sample collection at a specialized medical site, have a poor sample collection experience, make samples susceptible to contamination, and have difficulty in conveniently collecting the samples.

### SUMMARY OF THE INVENTION

In view of this, an objective of the present invention is to provide a new cervix sampler to solve the technical problems of a poor practical experience and difficult sample collection of an existing cervix sampler.

In order to solve the technical problems mentioned above, in an aspect, the present invention provides a cervix sampler, including:
a housing including a housing side wall and a closed housing distal wall, the housing side wall including a first notch, and the first notch being formed close to the housing distal wall; a sampling brush configured to be accommodated in the housing, at least a part of the sampling brush being exposed, in at least one state of use, to a position where the first notch is located; and a shaft including a shaft distal end and a shaft proximal end, the shaft distal end being connected to the sampling brush, the shaft proximal end further including a manipulator, and the manipulator, operated by an external force, driving the shaft and the sampling brush to rotate in a circumferential direction, so that the sampling brush acquires biological tissue in a state of use by being in contact with and rubbing against a target sample near the first notch.

In a further aspect, the present application provides an operating method of a cervix sampler, including:
providing a sampler according to any one of the foregoing embodiments, and setting a retaining wall to a first state of use; placing at least a part of the sampler at a position of a target sample; setting the retaining wall to a second state of use under the action of an external force, so that a sampling brush is exposed to a position where the first notch is located; and rotating a manipulator for at least one round under the action of an external force, so that the manipulator drives a shaft and the sampling brush to rotate in a circumferential direction, and the sampling brush acquires biological tissue by being in contact with and rubbing against the target sample near the first notch.

In a further aspect, the present application provides a cervix sampler kit, including:
a sampler according to any one of the foregoing embodiments; and
a container sized to accommodate at least a sampling brush therein.

In a further aspect, the present application provides a self-operated cervix cancer cell sampler, including:
a shaft including a shaft distal end and a shaft proximal end; a sampling brush configured to acquire a sample; a circumferentially rotatable manipulator having anti-skid stripes on an outer wall, the manipulator driving the shaft and the sampling brush to rotate in the same direction when the manipulator is rotated under the action of an external force; a housing including a housing side wall and a closed housing distal wall, the housing side wall including a first notch, and the first notch being formed close to the housing distal wall; the housing side wall being configured to include a second notch, and the position of the second notch corresponding to the position of the manipulator such that at least a part of the manipulator is exposed to a position where the second notch is located; an auxiliary plate fixedly connected to the housing; and a retaining wall connected to the auxiliary plate in a sliding manner to enable axial movement of the retaining wall relative to the housing; the retaining wall being sized and shaped to at least cover the first notch, and the retaining wall being configured to switch between a first state of use and a second state of use under the action of an external force; the retaining wall being configured to further include a window, and the position of the window corresponding to the position of the manipulator; wherein the sampling brush is configured to be accommodated in the housing, and at least a part of the sampling brush is exposed, in at least one state of use, to a position where the first notch is located; in the first state of use, the retaining wall covers the first notch; and in the second state of use, the retaining wall is away from the first notch, so that the sampling brush is exposed to the position where the first notch is located; and at least a part of the manipulator is exposed from the window; wherein the shaft distal end is connected to the sampling brush, and the shaft proximal end is connected to the manipulator; wherein the manipulator, operated by an external force, drives the shaft and the sampling brush to rotate in the circumferential direction for 5 rounds, so that the sampling brush acquires cervix cells in a state of use by being in contact with and rubbing against a target sample near the first notch.

Other exemplary embodiments are described herein.

The present invention has many advantages. In some embodiments, the sampler provided by the present invention has the characteristic of being easily pushed into vaginal and cervix target positions; in some embodiments, the sampler provided by the present invention has the advantage that the sampling brush can obtain a sample more easily and accurately; in some embodiments, the sampler provided by the present invention has the characteristic of being pushed forward or pulled backward more easily; and in some embodiments, the sampler provided by the present invention is ergonomically designed to have the advantage of being easier for sample collection and operation, and is suitable for self-service operation and use by a sampled person without the need for additional medical training. In some embodiments, the biological tissue collected by the sampler provided by the present invention is cervix cells, which can be particularly used for papanicolaou (PAP)/human papillomavirus (HPV) Co-testing for cervix cancer screening. According to the present application, the cervix sampler is suitable for use in medical places, and is also suitable for self-service use by the sampled person in non-medical places (for example, residences), making the process of sample collection more flexible and more protective of the sampled person's privacy and feelings of use. According to the present application, the cervix sampler makes it easier to obtain only the desired cervix cells in the process of sampling, and avoids obtaining other tissue or mucus, thereby avoiding an impure or contaminated sample and allowing for a more accurate cervix cancer screening result.

### BRIEF DESCRIPTION OF THE DRAWINGS

FIG. 1 is a schematic exploded diagram of a structure of a cervix sampler according to an embodiment of the present invention;

FIG. 2 is a schematic diagram of the embodiment as shown in FIG. 1 being in a first state of use;

FIG. 3 is a schematic diagram of the embodiment as shown in FIG. 1 being in a second state of use;

FIG. 4 is a schematic diagram of a partial structure of a sampling brush according to one embodiment.

### DETAILED DESCRIPTION OF EMBODIMENTS

In order to make the technical problems to be solved, the technical solutions, and the beneficial effects of the present invention clearer, the present invention will be described in further detail below with reference to the accompanying drawings and embodiments. It should be understood that the specific embodiments described herein are merely used to explain the present invention and are not intended to limit the present invention.

In the present invention, relative positions and directions of a container, such as upper, lower, top end, bottom end, inner and outer, are defined by taking the state where a cervix sampler is placed in normal use as the reference state.

In the present invention, the term “housing” refers to at least a part of the outermost structure of the sampler and is configured to protect the contents and provide a rigid profile for the sampler.

In the present invention, the term “axial direction” refers to a direction of the axis of a cylinder where the housing is located, with reference to the housing. In the present application, the axial direction and the axial direction of the sampler refer to the same direction.

In the present application, the term “circumferential direction” refers to a circumferential direction of the cylinder where the housing is located, with reference to the housing.

In the present application, the terms “proximal end and distal end” respectively refer to an end close to an operator's hand and an opposite end away from the operator's hand, with reference to the state of use of the sampler.

The cervix sampler of the present application may also be configured to sample vaginal biological tissue.

In the present invention, the term “comprising” means including the following elements, but not excluding other elements.

In an aspect, the present application provides a cervix sampler, including:
a housing including a housing side wall and a closed housing distal wall, the housing side wall including a first notch, and the first notch being formed close to the housing distal wall; a sampling brush configured to be accommodated in the housing, at least a part of the sampling brush being exposed, in at least one state of use, to a position where the first notch is located; and a shaft including a shaft distal end and a shaft proximal end, the shaft distal end being connected to the sampling brush, the shaft proximal end further including a manipulator, and the manipulator, operated by an external force, driving the shaft and the sampling brush to rotate in a circumferential direction, so that the sampling brush acquires biological tissue in a state of use by being in contact with and rubbing against a target sample near the first notch. The closed housing distal wall makes it easier for the sampler to move forward in the vagina under the action of a pushing force, and the sampling brush is in a sheltered position in the process of moving toward the inside of the vagina, and hence may not hinder the movement, so the sampling brush is better in user experience.

In at least one embodiment, the sampler further includes a retaining wall, the retaining wall being sized and shaped to at least cover the first notch, and the retaining wall being configured to switch between a first state of use and a second state of use under the action of an external force; in the first state of use, the retaining wall covers the first notch; and in the second state of use, the retaining wall is away from the first notch, so that the sampling brush is exposed to the position where the first notch is located. The sampling brush is exposed only after the sampler reaches a target cervical position, which makes a sample collected more accurate.

In at least one embodiment, the retaining wall of the sampler is configured to move in an axial direction along the housing side wall.

In at least one embodiment, the retaining wall of the sampler is configured to slide in the circumferential direction along the housing side wall.

In at least one embodiment, the retaining wall of the sampler is configured to further include a protrusion, and the retaining wall enables switching between the first state of use and the second state of use when the protrusion is operated by an external force.

In at least one embodiment, the manipulator of the sampler is a rotary converter, an outer wall of the rotary converter is provided with anti-skid stripes, and the manipulator drives the shaft and thus the sampling brush to rotate in the same direction when the rotary converter is rotated under the action of an external force. In one embodiment, the rotary converter is a dial rotary converter.

In at least one embodiment, the housing side wall of the sampler is configured to include a second notch, and the position of the second notch corresponds to the position of the manipulator such that at least a part of the manipulator is exposed to a position where the second notch is located; and the retaining wall is configured to further include a window, and the position of the window corresponds to the position of the manipulator when the retaining wall is in the second state of use such that at least a part of the manipulator is exposed outside.

In at least one embodiment, a proximal end of the retaining wall of the sampler is also provided with booster wings, and the booster wings are configured to apply an axial pushing force or pulling force to the retaining wall so as to enable switching between the first state of use and the second state of use.

In at least one embodiment, a proximal end of the housing of the sampler is also provided with a base, and a side of the base is rotationally connected to the shaft proximal end.

In at least one embodiment, a further side of the base of the sampler is configured to include a handle, and the handle is configured to apply an axial pushing force or pulling force to the sampler so that the sampler is placed into or withdrawn from a vagina.

In at least one embodiment, the housing distal wall of the sampler is substantially domed.

In at least one embodiment, the sampling brush of the sampler is detachably connected to the shaft distal end. The whole sampling brush is removed and placed into a sample collection container after the sampler completes sample collection, making sample acquisition very fast and easy.

In at least one embodiment, the sampler further includes an auxiliary plate fixedly connected to the housing; and the auxiliary plate is connected to the retaining wall in a sliding manner to enable axial movement of the retaining wall relative to the housing.

In at least one embodiment, the biological tissue collected by the sampler is cervix cells. The cervix cells are particularly used for papanicolaou (PAP)/human papillomavirus (HPV) Co-testing for cervix cancer screening.

In a further aspect, the present application provides an operating method of a cervix sampler, including:
providing a sampler according to any one of the foregoing embodiments, and setting a retaining wall to a first state of use; placing at least a part of the sampler at a position of a target sample; setting the retaining wall to a second state of use under the action of an external force, so that a sampling brush is exposed to a position where the first notch is located; and rotating a manipulator for at least one round under the action of an external force, so that the manipulator drives a shaft and the sampling brush to rotate in a circumferential direction, and the sampling brush acquires biological tissue by being in contact with and rubbing against the target sample near the first notch.

In at least one embodiment, the operating method further includes: setting the retaining wall to the first state of use again, and dragging a handle to withdraw the sampler from a vagina; and setting the retaining wall to the second state of use again, so that the sampling brush is exposed from the first notch, and the sampling brush is detached from a shaft distal end to acquire the collected biological tissue.

In at least one embodiment, the manipulator is rotated for at least 5 rounds to collect cervix cells.

In a further aspect, the present application provides a cervix sampler kit, including:
a sampler according to any one of the foregoing embodiments;
a container sized to accommodate at least a sampling brush therein.

In at least one embodiment, the kit further includes an auxiliary tool, the auxiliary tool being any one or more of a lubricant, tweezers and a biological tissue preservation solution. In one embodiment, a lubricant is applied to the housing distal wall to allow the sampler to be easily pushed into a target position inside the vagina; the biological tissue preservation solution is placed in the container in advance; and tweezers are used to assist in detaching the sampling brush including the sample, and placing the sampling brush in the container.

In a further aspect, the present application provides a self-operated cervix cancer cell sampler, including:
a shaft including a shaft distal end and a shaft proximal end; a sampling brush configured to acquire a sample; a circumferentially rotatable manipulator having anti-skid stripes on an outer wall, the manipulator driving the shaft and the sampling brush to rotate in the same direction when the manipulator is rotated under the action of an external force; a housing including a housing side wall and a closed housing distal wall, the housing side wall including a first notch, and the first notch being formed close to the housing distal wall; the housing side wall being configured to include a second notch, and the position of the second notch corresponding to the position of the manipulator such that at least a part of the manipulator is exposed to a position where the second notch is located; an auxiliary plate fixedly connected to the housing; and a retaining wall connected to the auxiliary plate in a sliding manner to enable axial movement of the retaining wall relative to the housing; the retaining wall being sized and shaped to at least cover the first notch, and the retaining wall being configured to switch between a first state of use and a second state of use under the action of an external force; the retaining wall being configured to further include a window, and the position of the window corresponding to the position of the manipulator; wherein the sampling brush is configured to be accommodated in the housing, and at least a part of the sampling brush is exposed, in at least one state of use, to a position where the first notch is located; in the first state of use, the retaining wall covers the first notch; and in the second state of use, the retaining wall is away from the first notch, so that the sampling brush is exposed to the position where the first notch is located; and at least a part of the manipulator is exposed from the window; wherein the shaft distal end is connected to the sampling brush, and the shaft proximal end is connected to the manipulator; wherein the manipulator, operated by an external force, drives the shaft and the sampling brush to rotate in the circumferential direction for 5 rounds, so that the sampling brush acquires cervix cells in a state of use by being in contact with and rubbing against a target sample near the first notch.

The various aspects of the present invention are further described below with reference to the accompanying drawings:

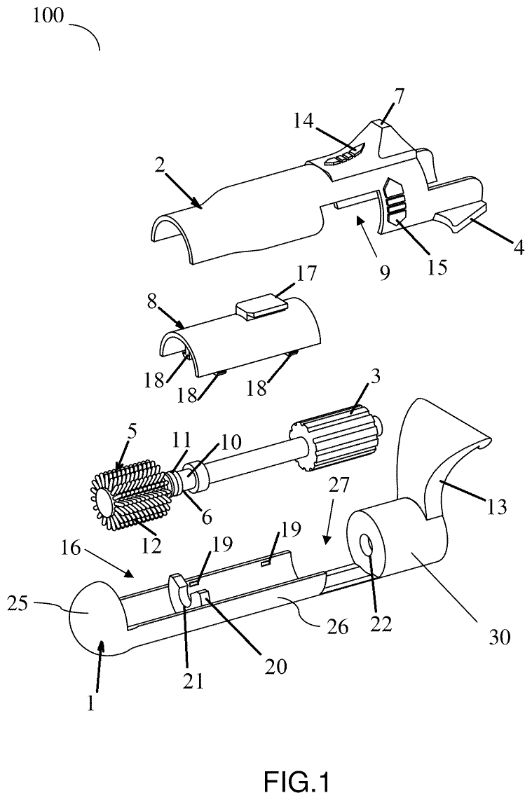

FIG. 1 is a schematic exploded diagram of a structure of an exemplary cervix sampler 100; in which the cervix sampler 100 includes a housing 1, a retaining wall 2, a shaft 10, a sampling brush 5, a base 30, a handle 13 and an auxiliary plate 8; wherein the housing 1 includes a housing side wall 26 and a closed housing distal wall 25, the housing side wall 26 being substantially a cambered surface on the whole, and the housing distal wall 25 being domed; the housing 1 is provided with a first notch 16 and a second notch 27, the first notch 16 being formed at a distal end of the housing 1, and the second notch 27 being formed at a proximal end of the housing 1; and an inner wall of the housing side wall 26 is provided with a backing ring 20, an external diameter of the backing ring 20 is equivalent to an internal diameter of the housing 1 at a corresponding position, and a through hole 21 is formed in the center of the backing ring 20 and is configured for the shaft 10 to pass through the backing ring 20 and for limiting the position of the shaft 10 in the housing 1. A plurality of bayonets 19 are arranged on an inner side of an edge of the housing side wall 26, fasteners 18 of which the number corresponds to that of the bayonets 19 are arranged on an inner side of an edge of the auxiliary plate 8, and after the fasteners 18 are combined with the bayonets 19, the auxiliary plate 8 is combined with the housing side wall 26 to form a substantially cylindrical body; the top of the auxiliary plate 8 is provided with a flange 17, a proximal end of the retaining wall 2 is provided with a protrusion 7, a space is formed in the bottom of the protrusion 7, the auxiliary plate 8 and the retaining wall 2 form a whole in a vertical direction when the flange 17 is embedded into the space formed in the bottom of the protrusion 7, and the flange 17 also forms a sliding track for the retaining wall 2 to move in an axial direction with respect to the auxiliary plate 8, so that the retaining wall 2 may move forward or backward in an axial direction of the housing 1; and the retaining wall 2 is configured to further include a front and rear indicating arrow 14, and the retaining wall 2 is moved forward or backward along the housing 1 when an external force drives the protrusion 7 according to the front and rear indicating arrow 14. The shaft 10 includes a shaft proximal end and a shaft distal end, the shaft proximal end is fixedly provided with a manipulator 3, and then the shaft proximal end forms a rotatable shaft connection with the base 30. Specifically, the base 30 is configured to include a shaft hole 22, and the shaft proximal end is inserted into the shaft hole 22 to form a rotatable shaft connection with the base 30; the shaft distal end is detachably connected to a sampling brush 5, and the sampling brush 5 includes bristles 12, a shaft 11 for connecting a brush head, and a connector 6; and the connector 6 is inserted into the shaft distal end of the shaft 10, i.e., connected to the shaft 10, and only when a certain external force is applied can the connector 6 be separated from the shaft 10. The manipulator 3 is cylindrical in shape, and an outer wall of the manipulator 3 is provided with anti-skid stripes. The position of the manipulator 3 corresponds to the position of the second notch 27 of the housing 1, and the position of the sampling brush 5 corresponds to the position of the first notch 16 of the housing 1. When an external force is applied to the manipulator 3 to make the manipulator rotate in the circumferential direction, the manipulator 3 drives the shaft 10 and the sampling brush 5 to rotate in the same direction, and the bristles 12 rub against the wall of the cervix and acquire cells on the inner wall of the cervix. The proximal end of the retaining wall 2 is also provided with a clockwise or counterclockwise indicating arrow 15 for indicating the direction of rotation of the manipulator 3. The proximal end of the retaining wall 2 also includes a window 9; and the proximal end of the retaining wall 2 is also provided with a pair of booster wings 4, the pair of booster wings 4 are arranged on two sides of the proximal end of the retaining wall 2, and the booster wing 4 has two stress surfaces, one for facilitating application of a pushing force and the other for facilitating application of a pulling force, so that the retaining wall switches between the first state of use and the second state of use. The proximal end of the sampler 100 further includes a handle 13, the handle 13 is connected to the sampler by means of the base 30, and the handle 13 is configured to push the sampler forward as a whole or pull the sampler backward as a whole, so that the sampler can be pushed into or withdrawn from the vagina.

In one embodiment, in the process of rotating the manipulator 3 to obtain the cervix cells, the manipulator 3 is rotated for at least 5 rounds.

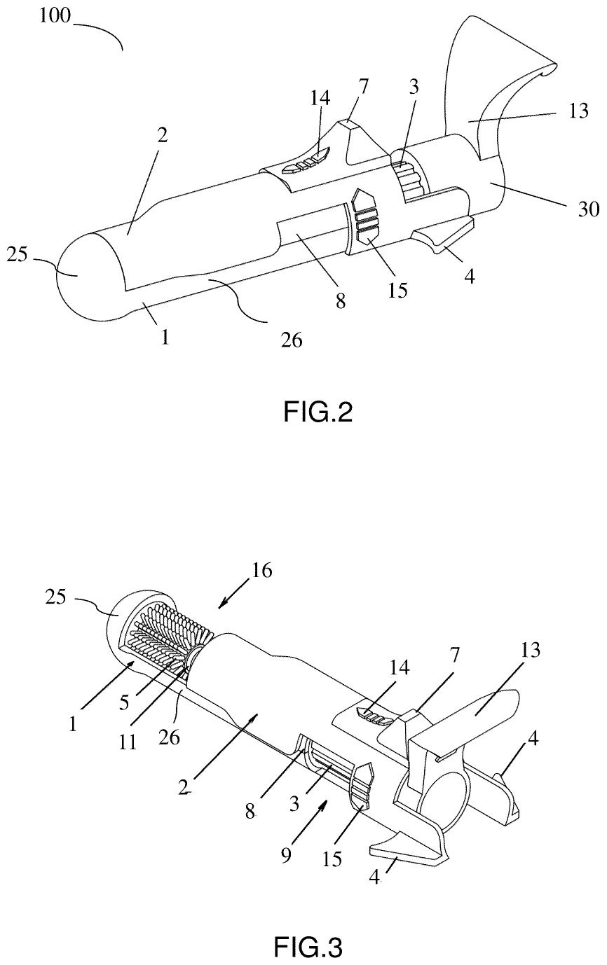

FIG. 2 is a schematic diagram of the cervix sampler 100 in a first state of use, the first state of use being the state of the sampler before and being pushed into the vagina, and the state of the sampler being withdrawn from the vagina upon completion of collection of the biological tissue; and the sampler 100 includes a housing 1, a retaining wall 2 is arranged on top of the housing 1, the housing 1 and the retaining wall 2 form a substantially smooth cylindrical shape in the first state of use, and the retaining wall 2 closes the first notch 16 of the housing 1, so that the sampling brush is closed inside the retaining wall 2, and the entry of the sampler into the vagina is smoother. In order to make the sampler enter the vagina more smoothly, a lubricant may also be applied to the outside of the housing 1. Upon completion of sample collection, the sampler switches to the first state of use again and then is taken out of the vagina, so as to prevent the sample from being contaminated during withdrawal, which may affect a laboratory report.

FIG. 3 is a schematic diagram of the cervix sampler 100 in a second state of use, the second state of use being a state in which the sampler 100 inside the vagina is ready to collect or is collecting a biological tissue sample. Under the action of a pulling force applied to the protrusion 7, the retaining wall 2 is moved toward the proximal end along the housing 1, so that the sampler is in the state shown in FIG. 3; and in the second state of use, the sampling brush 5 is exposed from the first notch 16 on the housing, and at the same time, the manipulator 3 is also exposed from the window 9 on the retaining wall 2. At this time, according to the direction indicated by the clockwise or counterclockwise indicating arrow 15, an operator circumferentially drives or rotates the manipulator 3 with an external force through the window 9 for many times, for example, 5 times, and the manipulator 3 drives the sampling brush 5 to rotate in the same direction, and the bristles 12 are in contact with and rub against the inner wall of the cervix from the space formed by the first notch, so as to collect and obtain the sample tissue from the inner wall of the cervix. Upon completion of sample collection, the sampler switches back to the first state of use as shown in FIG. 2, and then the sampler is removed from the vagina.

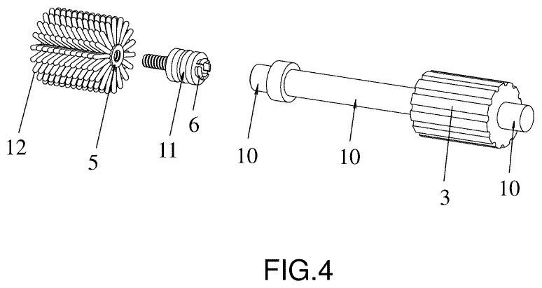

As shown in FIG. 4, which is a schematic diagram of a local structure of the sampling brush connected to the shaft, the sampling brush 5 is connected to the shaft 10 by means of the connector 6 as well as the shaft 11 for connecting the brush head, i.e., the connector 6 is inserted into the shaft distal end of the shaft 10. Upon completion of sampling, the operator applies an external force by clamping the shaft 11 with tweezers to pull the connector 6 out of the distal end of the shaft 10 so as to remove the sampling brush, and then places the sampling brush as a whole into a sample collection container to be immersed in a biological tissue preservation solution inside the container, for the next step of laboratory testing.

According to the present application, the cervix sampler is ergonomically designed for use by not only medical staff but also sampled persons for self-service operation and use. According to the present application, the cervix sampler is suitable for use in medical places, and is also suitable for self-service use by the sampled person in non-medical places (for example, residences), making the process of sample collection more flexible and more protective of the sampled person's privacy and feelings of use.

The preferred embodiments of the present invention are described above with reference to the accompanying drawings, and the scope of rights of the present invention is not limited thereby. Those skilled in the art can implement the present invention in many variation solutions without departing from the scope and essence of the present invention. For example, features of one embodiment can be used in another embodiment to obtain a further embodiment. Any modification, equivalent replacement and improvement made within the technical conception of the present invention shall fall within the scope of rights of the present invention.

1. A cervix sampler, comprising:
a housing, comprising a housing side wall and a closed, housing distal wall, the housing side wall comprising a first notch, and the first notch being formed close to the housing distal wall;
a sampling brush, configured to be accommodated in the housing, at least a part of the sampling brush being exposed, in at least one state of use, to a position where the first notch is located; and
a shaft, comprising a shaft distal end and a shaft proximal end;
wherein the shaft distal end is connected to the sampling brush, the shaft proximal end further comprises a manipulator, and the manipulator, operated by an external force, drives the shaft and the sampling brush to rotate in a circumferential direction, so that the sampling brush acquires biological tissue in a state of use by being in contact with and rubbing against a target sample near the first notch.

2. The sampler according to claim 1, further comprising a retaining wall, the retaining wall being sized and shaped to at least cover the first notch, and the retaining wall being configured to switch between a first state of use and a second state of use under the action of an external force;
wherein
in the first state of use, the retaining wall covers the first notch; and
in the second state of use, the retaining wall is away from the first notch, so that the sampling brush is exposed to the position where the first notch is located.

3. The sampler according to claim 2, wherein the retaining wall is configured to move in an axial direction along the housing side wall.

4. The sampler according to claim 2, wherein the retaining wall is configured to slide in a circumferential direction along the housing side wall.

5. The sampler according to claim 2, wherein the retaining wall is configured to further comprise a protrusion, and the retaining wall enables switching between the first state of use and the second state of use when the protrusion is operated by an external force.

6. The sampler according to claim 1, wherein the manipulator is a rotary converter, an outer wall of the rotary converter is provided with anti-skid stripes, and the manipulator drives the shaft and thus the sampling brush to rotate in the same direction when the rotary converter is rotated in the circumferential direction under the action of an external force.

7. The sampler according to claim 1, wherein the housing side wall is configured to comprise a second notch, the position of the second notch corresponding to the position of the manipulator such that at least a part of the manipulator is exposed to a position where the second notch is located; and the retaining wall is configured to further comprise a window, the position of the window corresponding to the manipulator when the retaining wall is in a second state of use such that at least a part of the manipulator is exposed outside.

8. The sampler according to claim 2, wherein a proximal end of the retaining wall is further provided with a pair of booster wings, the booster wings being configured to apply an axial pushing force or pulling force to the retaining wall so that the retaining wall enables switching between the first state of use and the second state of use.

9. The sampler according to claim 1, wherein a proximal end of the housing is further provided with a base, and a side of the base is rotationally connected to the shaft proximal end.

10. The sampler according to claim 9, wherein another side of the base is configured to comprise a handle, the handle being configured to apply an axial pushing force or pulling force to the sampler so that the sampler is placed into or withdrawn from a vagina.

11. The sampler according to claim 1, wherein the housing distal wall is substantially domed.

12. The sampler according to claim 1, wherein the sampling brush is detachably connected to the shaft distal end.

13. The sampler according to claim 2, further comprising an auxiliary plate, fixedly connected to the housing; wherein the auxiliary plate is connected to the retaining wall in a sliding manner to enable axial movement of the retaining wall relative to the housing.

14. The sampler according to claim 1, wherein the biological tissue is cervix cells.

15. An operating method of a cervix sampler, comprising:
providing a sampler according to claim 1, and setting a retaining wall to a first state of use;
placing at least a part of the sampler at a position of a target sample;
setting the retaining wall to a second state of use under the action of an external force, so that a sampling brush is exposed to a position where the first notch is located; and
rotating a manipulator for at least one round under the action of an external force, so that the manipulator drives a shaft and the sampling brush to rotate in a circumferential direction, and the sampling brush acquires biological tissue by being in contact with and rubbing against the target sample near the first notch.

16. The operating method according to claim 15, further comprising:
setting the retaining wall to the first state of use again, and dragging a handle to withdraw the sampler from a vagina; and
setting the retaining wall to the second state of use again, so that the sampling brush is exposed from the first notch, and the sampling brush is detached from a shaft distal end to acquire the collected biological tissue.

17. The operating method according to claim 15, wherein
the manipulator is rotated for at least 5 rounds to collect cervix cells.

18. A cervix sampler kit, comprising:
a sampler according to claim 1; and
a container sized to accommodate at least the sampling brush therein.

19. The kit according to claim 18, further comprising an auxiliary tool, the auxiliary tool being any one or more of a lubricant, tweezers and a biological tissue preservation solution.

20. A self-operated, cervix cancer cell sampler, comprising:
a shaft, comprising a shaft distal end and a shaft proximal end;
a sampling brush, configured to acquire a sample;
a circumferentially rotatable manipulator, having anti-skid stripes on an outer wall, the manipulator driving the shaft and the sampling brush to rotate in the same direction when the manipulator is rotated under the action of an external force;
a housing, comprising a housing side wall and a closed housing distal wall, the housing side wall comprising a first notch, and the first notch being formed close to the housing distal wall;
the housing side wall being configured to comprise a second notch, and the position of the second notch corresponding to the position of the manipulator such that at least a part of the manipulator is exposed to a position where the second notch is located;
an auxiliary plate, fixedly connected to the housing; and
a retaining wall, connected to the auxiliary plate in a sliding manner to enable axial movement of the retaining wall relative to the housing; the retaining wall being sized and shaped to at least cover the first notch, and the retaining wall being configured to switch between a first state of use and a second state of use under the action of an external force; the retaining wall being configured to further comprise a window, and the position of the window corresponding to the manipulator;
wherein the sampling brush is configured to be accommodated in the housing, at least a part of the sampling brush being exposed, in at least one state of use, to a position where the first notch is located;
in the first state of use, the retaining wall covers the first notch;
in the second state of use, the retaining wall is away from the first notch, so that the sampling brush is exposed to the position where the first notch is located; and at least a part of the manipulator is exposed from the window;
wherein the shaft distal end is connected to the sampling brush, and the shaft proximal end is connected to the manipulator;
wherein the manipulator, operated by an external force, drives the shaft and the sampling brush to rotate in the circumferential direction for 5 rounds, so that the sampling brush acquires cervix cells in a state of use by being in contact with and rubbing against a target sample near the first notch.
